(19) United States
(12) Patent Application Publication
(10) Pub. No.: US 2012/0265161 A1
(43) Pub. Date: Oct. 18, 2012

(54) FLUSHABLE BODY FLUID ABSORBENT COMPOSITE

(76) Inventors: John M. Banks, Guangzhou (CN); Qian (Michelle) Yang, Maidenhead (GB)

(21) Appl. No.: 13/503,840

(22) PCT Filed: Jul. 7, 2009
(86) PCT No.: PCT/CA2009/000940

§ 371 Date: Apr. 24, 2012

Related U.S. Application Data
(60) Provisional application No. 61/134,353, filed on Jul. 8, 2008.

(51) Int. Cl. A61L 15/62; A61L 15/24; A61L 15/28; A61L 15/26

(52) U.S. Cl. 604/372

(57) ABSTRACT

A flushable bodily liquid absorbent composite product, having a bodily liquid absorbent core, a moisture barrier layer applied to a garment side of the core, a film moisture break and a tissue backing layer. The tissue backing layer is readily soluble in water and the moisture barrier layer and the film moisture break are readily soluble in cold water. The use of the film moisture break is to absorb small amounts of moisture that is transferred through the moisture barrier in order to prevent weakening of the backing layer and prevent the transfer of positioning adhesive to the consumers garments. Preferred moisture barrier comprises polyvinyl alcohol and the preferred moisture break comprises carboxymethylcellulose or polyacrylimide.

### FIELD

The present invention relates to body fluid absorbent composite products such as women's undergarments, wound dressings, breast milk pads, adult incontinence pads, adult incontinence pouches, sanitary napkins, panty-liners, diapers, pet care products, training pads, and doggy diapers.

### BACKGROUND

Disposal of plastic infant diapers, and bodily fluid absorbent products in landfill sites is no longer a long term viable option. Often such materials constitute a biohazard risk and their disposal requires a minimum risk of human contact. Biodegrading these products first rather than allowing them to stay in their current form would be particularly beneficial to the environment.

One option that has been previously addressed in a number of patents has been the development of a composite product that can disperse in the sewerage system. Such products often rely on biodegradation of the barrier layer to disperse and, because of the time required to biodegrade, their disposal in waste water risks clogging of the sewerage system.

U.S. Pat. No. 6,217,562 issued to Brown et al. discloses a water-dispersible disposable enclosure such as an ostomy pouch which raises the temperature of the water that comes into contact with the pouch to a level at which the material melts, dissolves or otherwise disperses. This temperature elevation is accomplished by a water-activated exothermic reagent such as silica gel, aluminum chloride or calcium chloride. The water activated exothermic reagent is applied to the ostomy pouch by means of an external coating.

British Patent Application No. GB 2,295,553 discloses a bodily liquid absorbent core and a backing layer, which is applied to at least one face of the core. The backing layer is rapidly soluble in cold water but insoluble in viscous low volume bodily liquid discharges including menstrual fluid, blood and breast milk.

A layer, which is water soluble, covered by a water insoluble coating forms the backing layer, which is designed to prevent body fluids from escaping into the garment area. When the composite product is deposited in wastewater, the backing layer is exposed on its unprotected side and rapidly disperses in the water. One problem is that the backing layer is unprotected from penetration and degradation due to moisture from the garment side. Humidity at that location can be as high as 100% with temperatures up to 37° C.

U.S. patent application Ser. No. 10/905,636 also discloses a water soluble backing layer designed to prevent body fluids from escaping into the garment area. One problem found using this method is slow and intermittent moisture transfer through the insoluble layer weakening the water-soluble material prematurely and causing the transfer of positioning adhesive to the consumers garments.

Accordingly, it is an object of the invention to provide a composite product that can disperse rapidly in waste water, be resistant to not only body fluids on the body side of the composite product and resistant to moisture on the garment side but also resist degradation from the presence of moisture, and prevent the transfer of positioning adhesive to the consumers garments. It is a further object to provide such a composite product that is inexpensive.

### SUMMARY OF THE INVENTION

According to the invention there is provided a flushable bodily liquid absorbent composite product, having a bodily liquid absorbent core, attached to a moisture barrier, which in turn is attached to a moisture break having absorbent material held in place by a tissue backing layer that is applied to the consumers garments.

The backing layer is tissue of sufficient strength to retain its integrity when the composite product is removed from the consumer's clothes.

The moisture barrier is made up of polyvinyl dylene chloride of sufficient thickness to substantially make it impervious to moisture, attached to a layer of formulated polyvinyl alcohol. The polyvinyl alcohol is hydrolyzed to a range of 71 to 99.8%.

The polyvinyl alcohol layer may have a thickness of 20 to 35 microns while the polyvinyl dylene chloride layer may have a density of 2.2 to 4.0 grams per square meter (gsm).

Alternatively the polyvinyl dylene chloride may be omitted and the polyvinyl alcohol can include certain polymer additives to prevent, resist or slow degradation following exposure to bodily fluids. For example a polymer such as polycaprolactone can improve resistance to human blood based bodily fluids.

Positioning adhesive placed on the backing layer retains the product in place on the consumers clothing.

### BRIEF DESCRIPTION OF THE DRAWINGS

Further features and advantages will be apparent from the following detailed description, given by way of example, of a preferred embodiment taken in conjunction with the accompanying drawings, wherein:

FIG. 1 is a cross-sectional view of the composite product;

FIG. 2 is a cross-sectional view of an alternative product;

FIG. 3 is a cross sectional view of an alternative product.

### DETAILED DESCRIPTION WITH REFERENCE TO THE DRAWINGS

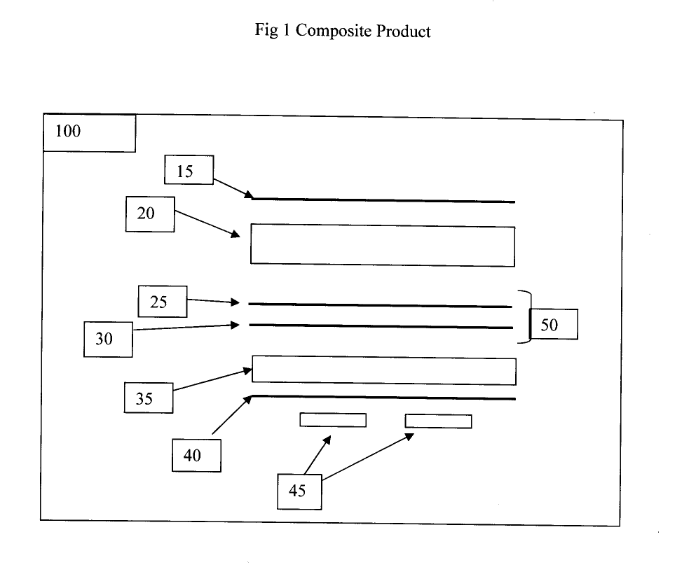

Referring to FIG. 1, the composite product 100 consists of a core 20 of absorbent material such as fluff pulp and carboxymethylcellulose. The absorbent pad of material 20 is covered by a coverstock web 15 on its body contacting side and a moisture barrier layer 50 on its garment facing side. The coverstock web 15 is made of a biodegradable material, such as rayon, viscose or polyvinyl alcohol so that it biodegrades after disposal. The moisture barrier layer 50 is made of two layers 25 and 30. The first layer 25 next to the core is made of 2.2 to 4.5 gsm of polyvinyl dylene chloride. One source of PVDC (F310) is Dow Plastics, a division of the Dow Chemical Company. The PVDC F310 can be applied by a flexographic printer as a solid solution in solvent. Upon evaporation of the solvent, a water impervious layer is formed. Usually two or more applications are required as the first develops pinholes that compromise the integrity of the film.

Other available PVDC formulations from other suppliers can be used that can be extruded or co-extruded with other layers.

The second layer 30 is formulated polyvinyl alcohol (PVA) with the base polyvinyl alcohol resin hydrolyzed to a range of 71 to 99.8%. This allows for it to be sufficiently soluble in cold water. A PVA film with a hydrolization of 89 to 99.8% is fully hydrolyzed. Hydrolization in these upper ranges work but a lower hydrolization ensures a more rapid dissolution in colder water while higher hydrolization can provide greater durability in warmer climates. The polyvinyl alcohol film may be cast or extruded to a thickness of between 20 to 38 microns.

A moisture break 35 consisting of absorbent material such as carboxymethylcellulose or polyacrylimide that can be held in place by materials such as tissue or fluff pulp form an absorbent area to absorb small amounts of moisture that is transferred through the moisture barrier.

A backing layer 40 is tissue paper to contain the moisture break absorbent material. The tissue paper is of sufficient dry strength to retain its integrity when removed from the consumers clothes but a low wet strength to disperse when flushed in a waste water system.

Finally, a positioning adhesive 45 can be applied to the backing layer 40. The glue adheres to the clothing of the user and helps keep the composite product in place. For applications such as adult incontinence and feminine hygiene positioning glue may be used. However, diapers and other applications would not ordinarily use positioning glue.

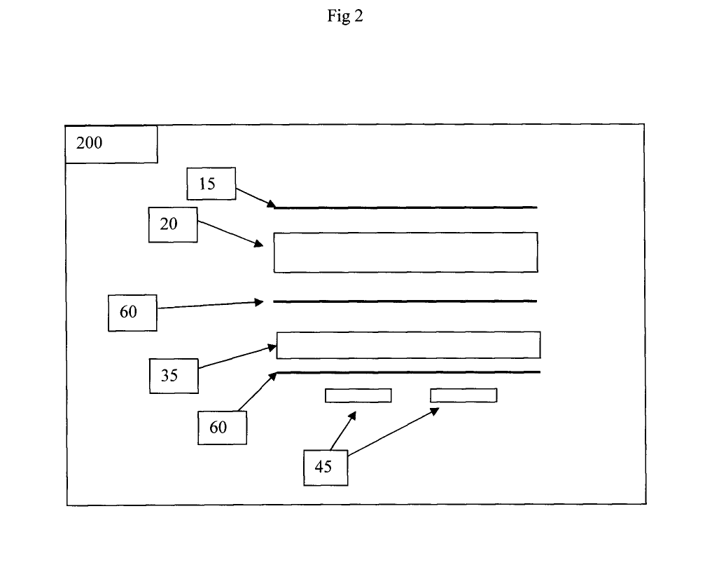

Referring to FIG. 2, the composite product 200 has layering different to FIG. 1 as a polyvinyl alcohol film formulation 60 is used as both the barrier layer and the backing sheet. This formulation includes a polymer to delay film breakdown and moisture transfer from bodily fluids, such as polycaprolactone as one additive in a formulation to slow polyvinyl alcohol degradation in the presence of blood.

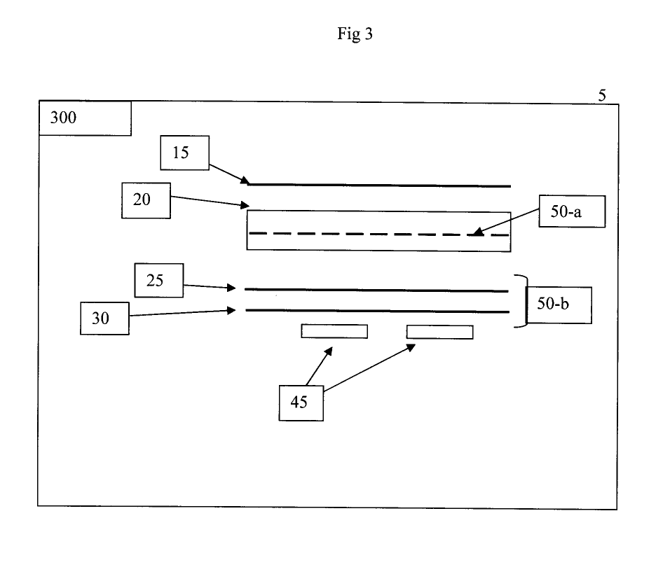

Referring to FIG. 3 the composite product 300 has one layer of polyvinyl dylene chloride and one layer of formulated polyvinyl alcohol together, although in this embodiment these films are embedded within the core of absorbent material 50-a. Also in this embodiment a similar two-layer film 50-b forms the backing layer to which the position adhesive is attached.

As an alternative, in FIG. 1 a clear see-through wing can be produced so the consumer does not have unsightly wings showing on the outside of their undergarments while simultaneously concealing body fluids contained in the body fluid absorbent core. In the embodiment 100 the two films 25 and 30 can be made colorless to create see-through wings, while the tissue backing layer 40 and moisture break 35 provide a white material area that conceals any bodily fluid held in the absorbent core 20. A similar product differentiation can be created in the other embodiments.

Accordingly, while this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications or embodiments as fall within the true scope of the invention.

We claim:

1. A flushable bodily liquid absorbent composite product, comprising:
(a) a bodily liquid absorbent core;
(b) a moisture barrier layer of polyvinyl alcohol applied to a garment side of said core;
(c) a backing layer of tissue paper, and
(d) a moisture break of absorbent material selected from the group comprising carboxymethylcellulose and polyacrylimide.

2. The product according to claim 1, wherein said moisture barrier layer is formulated polyvinyl alcohol hydrolyzed to a range of 71 to 99.8%

3. The product according to claim 1, wherein said moisture barrier comprises a first layer of polyvinyl dylene chloride of sufficient thickness to make it water impervious, and a second layer of a water soluble polymer.

4. The product according to claim 1, wherein the product further comprises wings constructed of a see-through material.

5. The product according to claim 3 wherein the second layer has a thickness of 20 to 35 microns.

6. The product according to claim 3, wherein the first layer has a density of 2.2 to 4.0 grams per square meter.

7. The product according to claim 1, wherein a positioning glue is applied to the backing layer of tissue paper.
